(19) United States
(12) Patent Application Publication
(10) Pub. No.: US 2010/0303770 A1
(43) Pub. Date: Dec. 2, 2010

(54) METHODS FOR CULTURING DERMAL CELLS FOR TREATMENT OF SKIN INJURIES SUCH AS BURNS

(76) Inventors: John Maslowski, Pottstown, PA (US); Myrna F. Thomas, Hainesport, NJ (US); Marie A. Lindner, Radnor, PA (US)

Correspondence Address: FISH & RICHARDSON P.C., P.O. BOX 1022, MINNEAPOLIS, MN, 55440-1022, US

(21) Appl. No.: 12/521,262

(22) PCT Filed: Dec. 27, 2007
(86) PCT No.: PCT/US07/88950

§ 371 Date: Aug. 2, 2010

Related U.S. Application Data
(60) Provisional application No. 60/882,274, filed on Dec. 28, 2006.

(51) Int. Cl. A61K 35/36; A61P 17/02; C12N 5/07

(52) U.S. Cl. 424/93.7; 435/379; 435/325; 435/408

(57) ABSTRACT

The present invention relates to novel methods of growing or otherwise producing unpassaged or minimally-passaged dermal cells from a small biopsy specimen for treating skin injuries significantly larger than the biopsy.

### FIELD OF THE INVENTION

The present invention relates to novel methods of culturing and growing dermal cells, and methods of use thereof to promote healing of burns and burn scars in an animal.

### BACKGROUND OF THE INVENTION

The skin is the largest organ of the human body. It consists of two main layers: the epidermis is the outer layer, sitting on and nourished by the thicker dermis. These two layers are approximately 1-2 mm thick. The epidermis consists of an outer layer of dead cells, which provides a tough, protective coating, and several layers of rapidly dividing cells called keratinocytes. The dermis contains the blood vessels, nerves, sweat glands, hair follicles, and oil glands. The dermis consists mainly of connective tissue, primarily the protein collagen, which gives the skin its flexibility and provides structural support. Fibroblasts, which make collagen, are the main cell type in the dermis.

Skin protects the body from fluid loss, aids in temperature regulation, and helps prevent disease-causing bacteria or viruses from entering the body. Skin that is damaged extensively by burns or non-healing wounds can compromise the health and well-being of the patient.

The American Burn Association reports that, each year in the United States, 1.1 million burn injuries require medical attention. Approximately 45,000 of these require hospitalization, and roughly half of those burn patients are admitted to a specialized burn unit. Approximately 5,500 die from these burns. In the UK, 13,000 patients are hospitalized annually with burns. According to the NIH, improving methods of wound healing and tissue repair offers tremendous opportunities to enhance the quality of life for trauma and burn patients, and may also help to reduce health care costs.

Significant partial and full thickness burns require hospitalization for fluid restoration, debridement, prevention and treatment of infections, and wound care which includes removal of necrotic tissue and wound coverage, to prevent infection, as well as to promote healing. Depending on the depth and size of a burn wound, a patient may require skin grafting of various kinds. The trend is for patients with serious burns to undergo grafting within the first 2-6 weeks of care, though it may often be repeated during recovery, and surgery to remove hypertrophic scars may be used later. Usually, the scars will mature and remodel during the first year. During that time, restrictive garments and extensive physical therapy are used to reduce restriction of mobility and hypertrophic scarring, but these tactics are often less than optimally successful, and frequently lead to disfiguring hypertrophic scars which may greatly restrict movement.

Wounds that are left to heal on their own can contract, often resulting in serious scarring; and if the wound is large enough, the scar can actually prevent movement of limbs. Skin grafting is a surgical procedure by which skin is placed over a burn or non-healing wound to permanently replace damaged or missing skin, or to provide a temporary wound covering. Wounds such as second or third-degree burns must be covered as quickly as possible to prevent infection or loss of fluid. Skin grafting also requires the tissue for grafting and the recipient site be as sterile as possible to prevent later infection that could result in failure of the graft.

Skin for grafting can be obtained from another area of the patient's body, called an autograft, if there is sufficient undamaged skin available, and if the patient is healthy enough to undergo the additional surgery required. Alternatively, skin can be obtained from another person (donor skin from cadavers is frozen, stored, and available for use), called an allograft, or from an animal (usually a pig), called a xenograft. Allografts and xenografts provide only temporary covering—they are rejected by the patient's immune system within seven (7) to ten (10) days and must be replaced with an autograft.

A split-thickness skin graft generally uses the epidermis and a small portion of the dermis, and usually adheres to the wound site within several days. For a split-thickness graft to be successful, the wound must not be too deep, because the blood vessels that will nourish the grafted tissue will come from the dermis of the wound itself.

A full-thickness graft involves both layers of the skin. Full-thickness autografts provide better contour, more natural color, and less contraction at the grafted site. The primary disadvantage of full-thickness skin grafts is that the wound at the donor site is larger and requires more careful management; often a split-thickness graft must be used to cover the donor site.

A composite skin graft is sometimes used, consisting of combinations of skin and fat, skin and cartilage, or dermis and fat. Composite grafts are used where three-dimensional reconstruction is necessary. For example, a wedge of ear containing skin and cartilage can be used to repair the nose.

Finally, a full thickness flap may be used to cover large wounds when the vascular supply of the graft is necessary to prevent ischemic compromise of the tissue. Such a full thickness flap may be used in certain parts of the body where the vascular supply is limited, or where the flap must provide thickness in an area which typically is prone to pressure, such as the buttocks or hips.

Several artificial skin products are available for burns. Unlike allographs and xenographs, these products are not rejected by the patient's body and encourage the generation of new tissue. Artificial skin usually consists of a synthetic epidermis and a collagen-based dermis. This artificial dermis, the fibers of which are arranged in a lattice, acts as a template for the formation of new tissue. Fibroblasts, blood vessels, nerve fibers, and lymph vessels from surrounding healthy tissue cross into the collagen lattice, which eventually degrades as these cells and structures build a new dermis. The synthetic epidermis, which acts as a temporary barrier during this process, is eventually replaced with a split-thickness autograft or with an epidermis cultured in the laboratory from the patient's own epithelial cells.

Once a skin graft has been put in place, it must be maintained carefully both during and after healing to decrease the amount of contracture.

The risks of skin grafting include those inherent in any surgical procedure involving anesthesia. In addition, the risks of an allograft procedure include rejection and transmission of infectious disease.

Fibroblasts are connective-tissue cells involved in tissue repair. Fibroblasts synthesize a variety of compounds, including collagens, glycosaminoglycans, reticular and elastic fibers, and glycoproteins found in the extracellular matrix. When a tissue is injured, nearby fibroblasts migrate into the wound, proliferate, and produce large amounts of collagenous matrix, which helps to isolate and repair the damaged tissue. See, e.g., Alberts et al., Molecular Biology of the Cell, p. 987, 2nd ed., (1992).

Co-owned patents U.S. Pat. Nos. 5,591,444; 5,858,390; 5,660,850; 5,665,372; 6,432,710; and 6,878,383 (incorporated herein by reference in their entirety), broadly describe the repair of skin, bone, and other tissues prior to the advent of the growing of fibroblast cultures drawn from biopsies of patients in need of tissue repair to effect such repair. The aforementioned patents also disclose compositions and methods of growing and culturing passaged autologous fibroblasts and using such fibroblasts to repair skin, bone, and other tissues.

Fibroblasts take significant time to grow to sufficient numbers for use in treating skin injury, and the passaging of cell cultures required to generate such numbers yields fibroblasts which may suffer from decreased viability and effectiveness. Multiply passaging fibroblasts also suffers drawbacks from increased use of materials, increased cost, and increased opportunities for contamination of the cultures. The disclosure in co-owned U.S. Pat. Appln. Ser. No. 60/823,871, filed 29 Aug. 2006, entitled “Method for Culturing Minimally-Passaged Fibroblasts and Uses Thereof”, provides methods to generate such fibroblasts in sufficient numbers and effectiveness without a multiplicity of passages. There are many defects for which cultured fibroblasts are an effective treatment. Some burns, however, are best treated with a mixture of cells comprising fibroblasts and other dermal cells, such as keratinocytes, and the like; these other cells also benefit from being minimally-passaged.

Where a burn site is relatively small, a skin biopsy may be taken which matches the burn in size. However, taking a larger biopsy for larger burns presents additional problems: the larger biopsy itself results in the creation of another wound, and patients with a large percentage of the body which is burned may not have suitable sites for sufficient skin to be harvested for grafting. Since grafts may fail to heal in severely compromised patients, use of a skin graft does not guarantee healing and may simply result in the creation of additional wounds.

The art, therefore, is in need of methods to prepare compositions for the treatment and repair of burns, particularly where the burns are larger than recommended skin graft biopsies. The present invention provides such a simplified method of preparing such compositions.

### SUMMARY OF THE INVENTION

In a surprising discovery, the inventors herein have found that a composition for the treatment of burns may be prepared from a small skin biopsy in size from about 1-4 inches, and between 4 and 12 cm² in area, taken with a dermatome, and comprising epidermis and at least partial dermis. Unlike an ordinary skin graft, the biopsy specimen taken from another part of the body may be significantly smaller than the burn area to be treated. Thus, a relatively small biopsy may be used to prepare a composition to treat much larger burns.

In one aspect, the biopsy specimen is broken into smaller pieces with a disposable micro tissue grinder and digested or partially digested with a dissociative or digestive enzyme, resulting in a variety of sizes of tissue and free cells comprising on the order of 60 million cells. These cells include a variety of cell types, including keratinocytes, fibroblasts, and other dermal cells. The tissue is then resuspended in growth medium and immediately transferred to a large flask, where it is cultured in the presence of growth medium. After a short culture duration, the tissue in the large flask comprises sufficient cells to be used to treat a burn area substantially larger than the original biopsy specimen.

In another aspect, the biopsy specimen is broken into smaller pieces, partially digested with a dissociative or digestive enzyme, followed by inactivation of the digestive enzyme, and the addition of phosphate buffered saline (PBS) for resuspension. In this aspect, the cells are also a variety of types, including keratinocytes, fibroblasts, and other dermal cells. The tissue remains in the digestive medium for only a short time, up to about 30 minutes to 1 hour at which point the digestive enzyme is inactivated and is ready for transplanting to the burn area of the patient.

The invention further provides a method of maintaining the minimally passaged dermal tissue substantially free of immunogenic proteins present in the suspension. The present invention may use allogeneic tissue or autologous tissue. In the latter aspect, the composition is histocompatible with a subject, thereby avoiding elicitation of an immune response and inflammation in the tissues of the subject near the burn site.

In one aspect, the present invention uses gentamicin as an antibacterial agent, as well as amphotericin B as a fungicide, during the initial culture stage. In another aspect, the invention avoids the use of antibiotics in the subject, and hence prevents the emergence of antibiotic resistant pathogens and deleterious side effects associated with antibiotics in the subject.

The present invention is thus directed to, among other things, a method of generating at least 60 million dermal cells from a small biopsy specimen, comprising the steps of:
a) grinding the small biopsy specimen with a micro tissue grinder to form a ground specimen;
b) digesting the ground specimen with a dissociative or digestive enzyme to form a cell suspension; and
c) harvesting the dermal cells from the cell suspension.

The enzyme may be a collagenase, such as liberase, trypsin, or the like. The method of the invention is capable of generating at least 60 million dermal cells, and may be used to generate 200 million or more unpassaged dermal cells.

In another aspect, the invention is directed to a method of generating at least 60 million dermal cells from a small biopsy specimen, comprising the steps of:
a) grinding the small biopsy specimen with a micro tissue grinder to form a ground specimen;
b) digesting the ground specimen with a dissociative or digestive enzyme to form a cell suspension;
c) culturing the cells in the cell suspension for a period from 3 to 14 days; and
d) harvesting the dermal cells.

In this aspect, as many as 300 million, or 450 million, or 600 million, or more unpassaged, or in some aspects, minimally-passaged, dermal cells may be generated.

The invention is also directed to a method of treating a skin injury in an animal, comprising the step of administering to the animal the dermal cells produced by other aspects of the invention. The skin injury may be a burn or other skin defect.

In this aspect, one or more administrations of dermal cells each comprises between 10 and 600 million minimally-passaged dermal cells, and the route of administration may be injection, or topical application into the skin injury area or area subadjacent to the skin injury.

Compositions comprising the dermal cells produced by the methods of the invention are also included. Further, conditioned medium produced by the methods of the invention is also included.

Each method of the invention yields a composition of cells suitable for use in treatment of burns and other skin injuries, and such compositions are within the scope of the invention.

Other objects and advantages of the present invention will become apparent to those skilled in the art from a review of the ensuing description.

### DETAILED DESCRIPTION OF THE INVENTION

The present invention is based on the surprising and unexpected discovery that biopsy-derived dermal tissue may be seeded and optionally cultured to numbers sufficient for exogenous administration to a patient to promote healing of tissue, without requiring multiple passaging of cultured cells. In the past, such biopsy-derived tissue was either directly used in a grafting procedure, or was digested to release free cells which were then cultured over a period of time. Such culturing required relatively high density seeding in smaller tissue culture flasks, followed by multiple passages into sequentially larger flasks, in order to generate a sufficient number of fibroblast cells. In co-owned U.S. Pat. Appln. Ser. No. 60/823,871, a method of generating fibroblasts with minimal passaging was disclosed; however, where a variety of dermal cell types are desired, the present invention provides a rapid means of generating sufficient tissue to treat a burn, and with minimal passaging of such cells.

As used herein, a “small biopsy” is one which is 1-5 mm thick, and between about 4 and about 12 cm² in area. Such a biopsy will be sufficient to treat burns up to about 20-50 times its area. In one embodiment of the invention, the target burn site is only 1-10 times the area of such a biopsy, in which case the biopsy tissue may be treated with dissociative or digestive enzyme, and suspended in PBS and antibiotics. Inactivation of the enzyme at 5 to 60 minutes is sufficient to generate a tissue composition suitable to treat such a burn site. In another embodiment, the tissue is used to initiate a culture, which is then cultured until desired cell numbers are achieved.

In another embodiment, wherein the burn site is substantially larger than a small biopsy, a small biopsy specimen is first broken into smaller pieces with a disposable micro tissue grinder, and digested or partially digested with a dissociative or digestive enzyme, yielding a variety of sizes of tissue and free cells comprising from about 25-50 million cells. The cells are also a variety of types, including keratinocytes, fibroblasts, and other dermal cells. The tissue is then transferred to a large flask with growth medium, where it is cultured in the presence of such growth medium. After a short culture duration ranging from about 3-14 days, the tissue in the large flask comprises sufficient cells to be used to treat a burn area substantially larger than the original biopsy specimen.

In brief, the method of the invention may be performed as follows: Initially, a small 1 to 5 mm thick biopsy specimen is taken with a dermatome, about 4-12 cm² in area, comprising epidermis and at least partial dermis. The biopsy specimen is cut into smaller pieces and transferred to a 50 mL conical tube containing a disposable micro tissue grinder Following grinding, the tissue is washed several times in about 30 mL (depending on the dermatome size) of wash media comprising IMDM medium with antibiotic agents, such as gentamicin (antibacterial) at a concentration of between 0.2-0.4 μg/mL, preferably about 0.3 μg/mL, and amphotericin B (antifungal) at a concentration of between 0.02-0.04 μg/mL, preferably about 0.03 μg/mL Each washing step generally comprises adding the wash media, keeping the tissue submerged from about 4 to 6 minutes, and removing the wash media.

Following the washing procedure, the tissue is then digested using a solution of a dissociative or digestive enzyme, incubated on an orbital shaker and then vortexed. In one embodiment, the enzyme is trypsin. In another embodiment, the enzyme is a collagenase enzyme, preferably liberase. Digestion time depends on which enzyme is used. In the case of liberase, digestion is allowed to proceed for about thirty minutes to one hour.

Growth medium is than added to neutralize the enzyme (when such neutralization is necessary), and the cells are centrifuged. Preferably, growth medium comprises IMDM (containing HEPES and L-glutamine and the aforementioned antibiotics) and 10% fetal bovine serum (FBS), although variations in growth medium will be appreciated by those of skill in the art. However, in embodiments in which the cell preparation is to be administered without culturing, the enzyme may be neutralized with a growth medium comprising the patient's own serum rather than FBS, or other neutralizers such as EDTA, cystine, and the like.

In one embodiment of the present invention, the pieces of tissue and free cells are allowed to rest for a brief period of time, from about 5 to about 30 minutes, at which point they may be directly administered to the burn area of a patient. These cells have not been passaged at all. The advantage conferred by this embodiment is that the combination of cells and tissue pieces is capable of treating a far larger burn area than the small biopsy would otherwise have been able to treat using traditional techniques. Where still more cells are required, they may be generated by the following further steps.

The digested mixture of cells is pelleted, resuspended in growth medium, and pipetted into a 2 liter media bottle to enable uniformly distributed transfer to a multilayer culture stack, such as a 5-layer or 10-layer culture stack. The multilayer culture stack is then incubated between 35-39° C. with about 4-6% CO₂. Additional growth medium may be added as needed, and conditioned medium is removed from the growing culture and may be stored for later use. Such supplementation of the flask with additional growth medium may be performed at intervals as needed, usually about every 3-7 days.

When the cells in the multilayer culture stack have reached about 90-100% confluence, which occurs in about seven days, they are harvested, generally yielding at least 1.0×10⁸ cells, preferably at least 2.0×10⁸ cells, more preferably at least 3.0×10⁸ cells. These cells would be characterized as unpassaged. The cells may then be shipped directly to the point of treatment location, as fresh cells, or may be cryopreserved, stored, and shipped at a later date. Preferably, the cells will be suspended in 10-20 mL of freezing medium (as described below), and transferred to freezing vials, 1.2 mL of suspension per vial. Each vial will thus contain about 2.2×10⁷ cells, more than a sufficient number for injection or other administration to a patient. Generally, an injection or topical administration treatment comprises from about 1-2×10⁷ cells fresh or cryopreserved cells. Numerous methods for successfully freezing cells for later use are known in the art and are included in the present invention.

Aliquots of about 1.2 mL of fresh (or previously frozen and thawed) suspension may be reseeded into a new flask, or a 5- or 10-layer culture stack if more cells are needed, or a stock of cells for the intended patient are to be maintained, and the above procedure is followed until harvest of the stack. Such reseeding of cell suspension will preferentially generate fibroblasts. When reseeding for the purposes of growing fibroblasts, any tissue culture technique that is suitable for the propagation of dermal fibroblasts from biopsy specimens may be used to expand the cells to practice the invention as described above, maintaining a low number of passages. Techniques well known to those skilled in the art can be found in R. I. Freshney, Ed., Animal Cell Culture: A Practical Approach (IRL Press, Oxford, England, 1986) and R. I. Freshney, Ed., Culture Of Animal Cells: A Manual Of Basic Techniques, Alan R. Liss & Co., New York, 1987), which are hereby incorporated by reference. If desired, the fibroblasts may be minimally passaged fibroblasts as described in co-owned U.S. Pat. Appln. Ser. No. 60/823,871.

The harvested cells may be frozen in any freezing medium suitable for preserving fibroblasts. In one embodiment, freezing medium comprises by volume about 70% growth medium, about 20% FBS and about 10% dimethylsulfoxide (DMSO); however, variations in the composition and proportions in the freezing media will be appreciated by those of skill in the art. In another embodiment, no FBS is used in the freezing medium which comprises, by volume, 50% IMDM, 42.5% cryopreservation solution such as ProFreeze™, and 7.5% DMSO. DMSO may also be substituted with, for example, glycerol. Thawed cells can also be used to initiate new cultures by following the methods of the invention as described above, directly seeding in a 10-layer culture stack, without the inconvenience of obtaining a second specimen.

The growth medium can be any medium suited for the growth of primary fibroblast cultures. The medium can be supplemented with human or non-human serum in an amount of between about 0.0% and about 20% by volume to promote growth of the fibroblasts. Higher concentrations of serum promote faster growth of the fibroblasts. Preferably, growth medium comprises IMDM (containing HEPES and L-glutamine) and 10% fetal bovine serum (FBS). In another embodiment, growth medium comprises glucose DMEM supplemented with about 2 mM glutamine, about 110 mg/L sodium pyruvate, about 10% (v/v) fetal bovine serum and antibiotics, wherein the concentration of glucose ranges from approximately 1,000 mg/L of medium to 6,000 mg/L of medium, and preferably is 4,500 mg/L.

Freshly harvested or thawed cells may be transported at 2-8° C., so long as they are utilized within 72 hours, preferably within 48 hours, and more preferably within 24 hours of their suspension. The cells may be suspended in an appropriate transport medium, a physiological solution with appropriate osmolarity, and may be tested for pyrogens and endotoxin levels. In another embodiment, the cells can be suspended in Krebs-Ringer solution comprising 5% dextrose or any other physiological solution. In a preferred embodiment, the transport medium is DMEM. Cryopreserved cells are preferably transported on dry ice.

The volume of saline or transport medium in which the cells are suspended is related to such factors as the number of fibroblasts the practitioner desires to inject, the extent of the defects to the subject's skin that are to be corrected, the size or number of the defects that are to be corrected, and the urgency of the subject's desire to obtain the results of the treatment. The practitioner can suspend the cells in a larger volume of medium and inject correspondingly fewer cells at each injection site.

Preferably, about 10-20 million cells are administered per administration. The number of cells administered in any given administration may need to be adjusted up or down depending upon several factors, including the size of the burn area to be treated, the potency of the fibroblasts within the cell population, and other considerations known in the art. Administrations are repeated as necessary until the desired result is achieved. The timing of a repeat administration, if necessary, is determined by periodic assessment by a physician.

The cells can be administered with other active agents as desired. For example, the cells can be administered in conjunction with basic fibroblast growth factor, which stimulates angiogenesis and is mitogenic for growth of keratinocytes and fibroblasts in vivo.

The method of the invention comprises (a) obtaining a sufficient number of dermal cells via biopsy and culture, and (b) administering the dermal cells to a burn site. The cells are prepared as a pharmaceutical composition, for direct injection, or may be delivered by other means, such as topically.

The administered cells may be allogeneic. In a preferred embodiment, the cells are histocompatible with the subject, that is, they are autologous cells that have been expanded by minimal passage in a cell culture system initiated by a biopsy specimen. In a preferred embodiment, the injected cells are dermal fibroblasts drawn from the subject to be treated.

Additionally, in some embodiments, the dermal cells may be combined with acellular matrices and/or filler materials, depending on the intended treatment area, as described in the co-owned patents cited above.

Conditioned medium stored during the practice of the method of the invention has many uses. For example, it may be used as a topical treatment for a variety of dermal defects, in conjunction with the administration of the cells grown by the method of the invention. Alternatively, the conditioned medium may be formulated into a composition suitable for topical administration without any cells.

Such conditioned medium generally comprises some or all of the growth factors or cytokines listed below in Table 1, depending on the stage in the procedure from which it was collected. The functions associated with these factors are also listed in Table 1.

TABLE 1

| Growth Factor/Cytokine | Function |
|---|---|
| Amino Acids | Important natural moisturizing factors |
| | (NMF) in the stratum corneum |
| | Promote skin smoothness |
| | Control skin pigmentation |
| | Components of collagen, which promotes |
| | elasticity and suppleness of skin |
| | Decrease the formation of keloid skin |
| | Component of peptides which have been |
| | shown to stimulate the natural rebuilding |
| | process without irritating the skin |
| Basic Fibroblast Growth | Promotes angiogenisis during wound |
| Factor (FGF basic) | healing |
| Collagen | Major structural protein in the skin |
| | Gives the skin its strength and durability |
| | and is responsible for the smooth, plump |
| | appearance of young, healthy skin |
| Endothelin-1 | Modulates collagen synthesis and the |
| | activities of MMP's and TIMP's |
| | Enhanses the release of Collagens I and III |
| | Decreases MMP-1 production |
| | Promotes fibroblast contraction |
| Eotaxin | Is a potent chemoattractant for other cell |
| | types, primarily eosinophils |
| Granulocyte-Colony | Stimulates the production of white blood |
| Stimulating Factor | cells |
| (G-CSF) | |
| Granulocyte Macrophage - | Stimulates stem cells to produce |
| Colony Stimulating Factor | granulocytes (neutrophils, eosinophils, |
| (GM-CSF) | and basophils) and macrophages as part |
| | of the immune/inflammatory response |
| Interleukin-6 | Stimulates the expression of MMP-1, |
| (IL-6) | TIMP-1, fibronectin, collagen, and |
| | glycosaminoglycan |
| | Promotes keratinocytes migration |
| | Promotes cutaneous wound healing |
| Interleukin-7 | Growth factor for both B- and T-cell |
| (IL-7) | lineages |
| Interleukin-8 | Important mediator of wound healing |
| (IL-8) | Inflammatory chemokine |
| Interleukin-18 | Inflammatory cytokine, which modulates |
| (IL-18) | MMP-2 production |
| | Is a potent chemoattractant for other cell |
| | types |
| Monocyte Chemoattractant | Is a potent chemoattractant for other cell |
| Protein-1 (MCP-1) | types |
| | Increases Type I collagen production |
| | Increases production of MMP-1 and |
| | TIMP-1 |
| Matrix Metalloproteinase-2 | Degrades extracellular matrix |
| (MMP-2) | components, and allows remodeling of |
| (also called Gelatinase A or | the extracellular matrix |
| Type IV Collagenase) | |
| Matrix Metalloproteinase-3 | Degrades extracellular matrix |
| (MMP-3) | components, and allows remodeling of |
| (also called Stromelysin 1 or | the extracellular matrix |
| Progelatinase) | |
| Plasminogen Activator | Promotes wound healing |
| Inhibitor-1 (PAI-1) | |
| Stem Cell Factor | Plays a key role in hematopoiesis |
| Tissue Factor | Promotes wound healing |
| Tissue Inhibitor of Matrix | Bind with MMP's to form complexes |
| Metalloproteinase-1 | wherein the MMP catalytic sites are |
| (TIMP-1) | unavailable to substrate |
| Vascular Endothelial Growth | Promotes proliferation of capillary |
| Factor (VEGF) | endothelial cells |

### Examples

The following Examples serve to illustrate the present invention and are not intended to limit its scope in any way.

### Example 1

### Composition from Small Biopsy Specimen

For Immediate Treatment

All procedures in this example were performed under aseptic conditions. Initially, a dermal fibroblast culture was initiated from a small 1 to 5 mm full thickness biopsy specimen from the skin of a pig, in area about 8 cm². The biopsy specimen was placed in a 50 mL conical tube and washed three times in a wash medium pre-warmed by incubation at 37.0±2.0° C. for 15 to 30 minutes. The wash medium comprised IMDM medium with gentamicin (antibacterial) at a concentration of 0.3 μg/mL and amphotericin B (antifungal) at a concentration of 0.03 μg/mL. For each wash, 20 mL of wash medium was added to the 50 mL conical tube, and the biopsy was maintained submerged for 4-6 minutes. The wash medium was then removed by pipette.

The washed biopsy specimen was then digested by pipetting 10 mL of a pre-warmed solution of liberase enzyme for about 60 minutes. The conical tube was then placed on an orbital shaker incubated at 37.0±2.0° C. at 100 rpm for about 60 minutes. The conical tube was then vortexed for 10 seconds.

20 mL of media containing a neutralizing agent was pipetted to the tube to neutralize the liberase enzyme and suspend the cells, and the cells were then pelleted in a centrifuge at 300×g (or about 1200 rpm) for 5 minutes at 5.0±3.0° C. The cells were then ready to be directly administered to the injury site of the patient.

### Example 2

### Composition from Small Biopsy Specimen

Initiation of Culture

All procedures in this example were performed under aseptic conditions. Initially, a dermal fibroblast culture was initiated from a small 1 to 5 mm full thickness biopsy specimen from the skin of a pig, in area about 8 cm². The biopsy specimen was placed in a 50 mL conical tube and washed three times in a wash medium pre-warmed by incubation at 37.0±2.0° C. for 15 to 30 minutes. The wash medium comprised IMDM medium with gentamicin (antibacterial) at a concentration of 0.3 μg/mL and amphotericin B (antifungal) at a concentration of 0.03 μg/mL. For each wash, 20 mL of wash medium was added to the 50 mL conical tube, and the biopsy was maintained submerged for 4-6 minutes. The wash medium was then removed by pipette.

The washed biopsy specimen was then digested by pipetting 10 mL of a pre-warmed solution of liberase enzyme for about 60 minutes. The conical tube was then placed on an orbital shaker incubated at 37.0±2.0° C. at 100 rpm for about 60 minutes. The conical tube was then vortexed for 10 seconds.

20 mL of growth medium was pipetted to the tube to neutralize the liberase enzyme and suspend the cells, and the cells were then pelleted in a centrifuge at 300×g (or about 1200 rpm) for 5 minutes at 5.0±3.0° C. Growth medium comprised IMDM (containing HEPES, L-glutamine, antibiotics gentamicin at a concentration of 30 mg/mL and amphotericin B at a concentration of 15 μg/mL), and 10% fetal bovine serum (FBS).

The supernatant was aspirated, and the cells were resuspended in 20.0 mL of growth medium. 1400 mL of growth medium was placed in a 2 liter media bottle and the resuspended cells were then pipetted into the media bottle. An additional 10.0 mL of growth media rinsed the 50 mL conical tube and was then added to the media bottle, which was rocked gently to distribute the cells evenly over the surface.

A 10-layer cell culture stack was prepared by replacing one of its standard caps with a universal cap. The cell suspension from the 2 liter media bottle was then slowly added to the 10-layer culture stack through a sterile funnel in the port, while the opposite standard cap was loosened to vent the stack, swirling the bottle at intervals during the pouring process to ensure capture of as many cells as possible. 100 mL of growth medium was added to the bottle to rinse its surface, and then added to the 10-layer culture stack. The universal cap was then replaced with a solid cap. The culture stack was tipped onto the side with the solid cap to allow media to level across all layers of the culture stack, and then tilted towards the end without caps and placed flat again, then gently rocked. Finally, the solid cap was replaced with the original standard cap, and the 10-layer culture stack was incubated at 37.0±2.0° C. with about 4-6% CO₂.

Fresh growth medium was added to the flask as needed by aspirating about half the volume of media and adding about 750 mL of fresh growth medium. The aspirated medium was saved as conditioned medium for later use.

Harvesting of Dermal Cells for Administration to Burn Site

When the cells in the 10-layer culture stack reached about 95-100% confluence, they were harvested, yielding about 3.0×10⁸ cells. First, 15 mL of spent growth media was aspirated, to which about 5×10⁶ harvested cells were added, to check for mycoplasma contamination. The remaining spent growth media was aspirated and saved as conditioned media. 600 mL of PBS was pipetted into the culture stack (300-600 mL may be used for this purpose), replacing its filter vent caps with solid caps, and the culture stack was tipped allowing the PBS to wash all layers of the stack, which was then incubated for 2-3 minutes per wash. The PBS was pipetted or aspirated off after each rinse. 300 mL of Trypsin-EDTA (200-500 mL may be used for this purpose) solution was pipetted into the culture stack and evenly distributed therein. The culture stack was then incubated for 4-6 minutes at 37.0±2.0° C.

When 80-100% of the cells “rounded up,” having a round appearance, the sides of the flask were tapped to release the cells into suspension. About 400 mL of pre-warmed growth medium was added and evenly distributed therein to neutralize the enzyme, and the cell suspension was then transferred to two 500 mL conical tubes, about 350 mL in each. 300 mL growth media as used to rinse the culture stack, which was then evenly divided between the two 500 mL conical tubes. The cells were then pelleted in a centrifuge at about 130-170×g for about 10 minutes at 5±3° C. The supernatant was aspirated. 30 mL of growth medium was used to resuspend the cells in one conical tube, then transferred to the other tube. 20 mL of growth medium was then used to rinse the first tube and then added to the other tube. Additional growth medium was then added to the tube containing the cell suspension to bring the total volume to 200 mL. Quality control samples were taken at this time, though they may be taken at any other time during the process.

The cells were then pelleted, the supernatant removed, and the cells resuspended in cold IMDM medium (5±3° C.) to a target concentration of 4.4×10⁷ cells/mL (generally between about 5-10 mL). The cell suspension was then stored in a refrigerator at 5±3° C. ready for shipping to the site of administration. Shipping will generally be performed in a 2-8° C. cold pack system sent to the practitioner for use. Alternatively, the cells may be shipped cryoperserved as described below, to be thawed at the site where they will be used.

Cryopreservation

To prepare the cells for cryopreservation, the cell suspension was diluted 1:1 with an equal volume of 2× concentrated freeze media comprising 85% ProFreeze and 15% DMSO, such that the final concentrations of ProFreeze and DMSO are 42.5% and 7.5% respectively. The volume of freeze media was prepared in a 15 mL tube, and then slowly pipetted dropwise into the cell suspension, allowing the freeze media to run along the side of the tube into the suspension. The tube was then pulse vortexed for 5 seconds, wiped with 70% isopropyl alcohol, then pipetted into cryovials, 1.2 mL or 0.6 mL per vial depending on volume requirements for testing and injection preparation. The suspension was swirled between each vial fill to ensure homogeneous cell suspension during the filling process. Each vial contained about 2.2×10⁷ cells, sufficient for injection or other administration into a patient.

Thawing the Preparation

Cells may be shipped fresh, or cryopreserved, or thawed from previous cryopreservation. To perform a thaw and preparation for two production injection vials, 3 vials were warmed to 37.0±2.0° C. until almost completely thawed. The thawed suspension was pipetted into a 50 mL conical tube pre-filled with 13 mL of PBS. Each vial was rinsed with another 1 mL of PBS which was then added to the tube. The cells were then pelleted (150×g for 10 minutes at 5±3° C.). The PBS was aspirated, the cells were resuspended in 17 mL of DMEM, then repelleted. The DMEM wash was aspirated, and then the cells were resuspended in 3.0 mL of DMEM. The cells were then ready for shipping in a 2-8° C. cold pack system to the practitioner for use. Alternatively, the cells could be cryoperserved and shipped at a later date, to be thawed at the site where they will be used.

The present invention is not to be limited in scope by the specific embodiments described above, which are intended as illustrations of aspects of the invention. Functionally equivalent methods and components are within the scope of the invention. Indeed, various modifications of the invention, in addition to those shown and described herein, will become apparent to those skilled in the art from the foregoing description. Such modifications are intended to fall within the scope of the appended claims. All cited references are, hereby, incorporated by reference.

1. A method of generating at least 60 million dermal cells from a small biopsy specimen, comprising the steps of:
a) grinding the small biopsy specimen with a micro tissue grinder to form a ground specimen;
b) digesting the ground specimen with a dissociative or digestive enzyme to form a cell suspension; and
c) harvesting the dermal cells from the cell suspension.

2. The method of claim 1, wherein the enzyme is a collagenase.

3. The method of claim 2, wherein the collagenase is liberase.

4. The method of claim 1, wherein the enzyme is trypsin.

5. The method of claim 1, wherein at least 200 million minimally-passaged dermal cells are generated.

6. A method of generating at least 60 million dermal cells from a small biopsy specimen, comprising the steps of:
a) grinding the small biopsy specimen with a micro tissue grinder to form a ground specimen;
b) digesting the ground specimen with a dissociative or digestive enzyme to form a cell suspension;
c) culturing the cells in the cell suspension for a period from 3 to 10 days; and
d) harvesting the dermal cells.

7. The method of claim 1, wherein at least 300 million minimally-passaged dermal cells are generated.

8. The method of claim 1, wherein at least 600 million minimally-passaged dermal cells are generated.

9. The method of claim 1, wherein the dermal cells have not been passaged.

10. The method of claim 1, wherein the dermal cells have been passaged no more than one time.

11. A method of treating a skin injury in an animal, comprising the step of administering to the animal dermal cells generated by the method of claim 1.

12. The method of claim 11 wherein the skin injury is a burn.

13. The method of claim 11, wherein one or more administrations each comprises between 10 and 600 million minimally-passaged dermal cells.

14. The method of claim 12, wherein the administration is by injection into the burn area or area subadjacent to the burn.

15. The method of claim 12, wherein the route of administration is topical application.

16. A composition comprising minimally passaged dermal cells generated by the method of claim 1.

17. A composition comprising conditioned medium produced by the method of claim 6.
